(19) United States
(12) Patent Application Publication
(10) Pub. No.: US 2024/0408329 A1
(43) Pub. Date: Dec. 12, 2024

(54) NASAL ASPIRATOR

(71) Applicant: Shenzhen Lute Jiacheng Supply Chain Management Co., Ltd., Shenzhen (CN)

(72) Inventor: Jieqing ZHENG, Shenzhen (CN)

(73) Assignee: Shenzhen Lute Jiacheng Supply Chain Management Co., Ltd., Shenzhen (CN)

(21) Appl. No.: 18/350,980

(22) Filed: Jul. 12, 2023

(30) Foreign Application Priority Data
Jun. 7, 2023 (CN) 202321451447.1

(51) Int. Cl. A61M 15/08; A61M 1/00

(52) U.S. Cl. CPC A61M 15/08; A61M 1/741; A61M 1/86

(57) ABSTRACT

Disclosed is a nasal aspirator, which includes a head portion provided with a first cavity inside, a reservoir connected to head portion and disposed in first cavity and provided with a second cavity inside, a drainage tube connected to head portion and extended from a first end of reservoir into second cavity and provided with a first air channel inside, an anti-reflux member abutted against a position between reservoir and head portion and/or a position between reservoir and drainage tube, and a negative pressure member. A third cavity is formed between reservoir and head portion. An end of anti-reflux member close to drainage tube is extended along an outer wall of drainage tube and towards a second end of reservoir, and the anti-reflux member is provided with a second air channel which communicates second cavity with third cavity. The negative pressure member is communicated with third cavity.

### CROSS-REFERENCE TO RELATED APPLICATIONS

This application claims priority to Chinese Patent Application No. 202321451447.1, filed on Jun. 7, 2023, the entire contents of which are incorporated herein by reference.

### TECHNICAL FIELD

The present application relates to the technical field of daily necessities, in particular to a nasal aspirator.

### BACKGROUND

A nasal aspirator is a commonly used and relatively simple tool for aspirating a nasal foreign body such as snot. The nasal aspirator is mainly used to clear the nasal foreign body for the user to breathe smoothly, such that the nasal symptoms caused by mild allergic rhinitis can be relieved.

The existing nasal aspirator usually has an anti-reflux structure, but the anti-reflux structure can avoid reflux when the main body of the nasal aspirator is in a vertical posture, with a poor anti-reflux effect. Once the nasal aspirator is accidentally knocked over and is in a horizontal posture, the nasal foreign body in the reservoir will flow to a side of the reservoir close to the nozzle, and then the nasal foreign body may reflux into the nozzle, and may also enter the negative pressure air channel or even the suction pump chamber. After the nasal foreign body refluxing into the nozzle is hardened, it will block the path that sucks the nasal foreign body, to make the suction function of the nasal aspirator poor. The nasal foreign body entering into the negative pressure air channel or the suction pump chamber will block the negative pressure air channel or abrase the power unit, the usage effect of the product is seriously affected and the service life of the product is reduced.

### SUMMARY

The main purpose of the present application is to provide a nasal aspirator, aiming to solve the technical problem in the related art that an anti-reflux structure of the nasal aspirator has a poor anti-reflux effect.

In order to achieve the purpose above, the present application provides a nasal aspirator which includes a head portion, a reservoir, a drainage tube, an anti-reflux member and a negative pressure member. The head portion is provided with a first cavity inside. The reservoir is connected to the head portion and is disposed in the first cavity, the reservoir is provided with a second cavity inside, and a third cavity is formed between the reservoir and the head portion. The drainage tube is connected to the head portion and is extended from a first end of the reservoir into the second cavity, and the drainage tube is provided with a first air channel inside. The anti-reflux member is abutted against a position between the reservoir and the head portion and/or a position between the reservoir and the drainage tube. An end of the anti-reflux member close to the drainage tube is extended along an outer wall of the drainage tube and towards a second end of the reservoir, and the anti-reflux member is provided with a second air channel which communicates the second cavity with the third cavity. The negative pressure member is communicated with the third cavity and is configured for an air suction to make the third cavity generate negative pressure, and the negative pressure is transmitted from the second air channel and the second cavity to the first air channel.

In an embodiment, the anti-reflux member includes a sleeve and an abutting ring. The sleeve is provided around and abutted against a periphery of the reservoir. The sleeve is extended towards the second end of the reservoir. An inner edge of the abutting ring is connected to the sleeve, and an outer edge of the abutting ring is abutted against the reservoir. Both the sleeve and the abutting ring are provided with the second air channel.

In an embodiment, both the sleeve and the drainage tube are extended towards the second end of the reservoir, and an end of the drainage tube close to the second end of the reservoir is extended out of an end of the sleeve close to the second end of the reservoir.

In an embodiment, the nasal aspirator includes a plurality of the second air channels, and the plurality of the second air channels are provided at intervals along a periphery of the anti-reflux member.

In an embodiment, the reservoir includes a first reserve portion and a second reserve portion. The second reserve portion is connected to a side of an end of the first reserve portion away from the drainage tube, and the second reserve portion is extended away from the drainage tube.

In an embodiment, the reservoir is provided at a middle of the first cavity, and the drainage tube corresponds to a center of the reservoir.

In an embodiment, the head portion includes an upper housing and a base. The first cavity is formed between the upper housing and the base. The negative pressure member is connected to the base. An air hole is formed at the base. The negative pressure member is communicated with the third cavity through the air hole. The nasal aspirator further includes a nozzle, the nozzle is mounted on a side of the upper housing away from the first cavity. The nozzle is provided with an air inflow channel communicated with the first air channel.

In an embodiment, the nozzle is detachably mounted on the upper housing.

In an embodiment, the nasal aspirator further includes a main body, the base is detachably mounted on the main body by a connecting structure. The negative pressure member is provided inside the main body. The negative pressure member includes an air pump and a suction pipe, an air suction channel is formed inside the suction pipe. The air pump is communicated with the air suction channel for the air suction, to make the air inflow channel generate negative pressure.

In an embodiment, the connecting structure includes a clamping block and a clamping groove. The clamping block is provided on the base, and the clamping groove is provided on the main body. The clamping groove includes an insertion portion and a clamping portion communicated with the insertion portion. The clamping block is configured to insert from the insertion portion and rotate to clamp with the clamping portion.

When using the nasal aspirator to aspirate a nasal foreign body such as snot, put the head portion into the nasal cavity, turn on the switch of the negative pressure member, and then the negative pressure member operates an air suction. Both the first air channel and the second air channel which are communicated with the negative pressure member generate negative pressure, and the nasal foreign body in the nasal cavity is detached from the nasal cavity and enters the first air channel due to the negative pressure in the first air channel. The nasal foreign body which has entered the first air channel flows into the reservoir through the first air channel due to gravity and the negative pressure in the first air channel, the nasal foreign body is stored in the reservoir at the end due to gravity, thereby realizing a nasal foreign body suction function of the nasal aspirator.

When the nasal aspirator is accidentally knocked over, it gradually changes from a vertical posture to a horizontal posture, the nasal foreign body in the reservoir will first flow along an inner wall of the reservoir. One end of the anti-reflux member abuts against the reservoir, so the nasal foreign body cannot flow out of the reservoir from the end of the anti-reflux member that abuts against the reservoir, then the nasal foreign body will flow along the anti-reflux member to the other end of the anti-reflux member. Since the other end of the anti-reflux member is extended towards a second end of the reservoir, so that the nasal foreign body flowing along the anti-reflux member cannot cross the end of the anti-reflux member close to the drainage tube, thereby preventing the nasal foreign body from flowing out of the reservoir, with a high anti-reflux effect. The nasal foreign body cannot reflux into the first air channel and blockage of the first air channel is prevented, the effect of sucking the nasal foreign body is improved. Meanwhile, the nasal foreign body either cannot enter the second air channel and the air inflow channel, which prevents the nasal foreign body from entering and blocking the second air channel and the air suction channel, thereby preventing the negative pressure member from abrasion, with a good usage effect of the product, and the service life of the product is increased. In addition, the second air channel that communicates the second cavity with the third cavity is opened on the anti-reflux member, such that there is no need to open a groove for passing air on the cavity wall of the first cavity, to facilitate the cleaning of the second air channel.

### BRIEF DESCRIPTION OF THE DRAWINGS

In order to more clearly illustrate the technical solutions in the embodiments of the present application or in the prior art, drawings used to illustrate the embodiments and the prior art are simply introduced below. Obviously, drawings introduced below are just some of the embodiments in the present application. For those skilled in the art, other figures may be further obtained without creative efforts according to the structures shown in drawings below.

FIG. 1 is a schematic structural view of a nasal aspirator according to an embodiment of the present application.

FIG. 2 is a partial schematic structural view of the nasal aspirator according to an embodiment of the present application.

FIG. 3 is a schematic structural view of an anti-reflux member of the nasal aspirator according to an embodiment of the present application.

FIG. 4 is a schematic structural view of a base and a part of a main body of the nasal aspirator according to an embodiment of the present application.

The realization of the purpose, the functional feature, and the advantage of present application will be further illustrated referring to the drawings.

### DETAILED DESCRIPTION OF THE EMBODIMENTS

The technical solutions of the embodiments of the present application will be described in more detail below with reference to the accompanying drawings. It is obvious that the embodiments to be described are only some rather than all the embodiments of the present application. All other embodiments obtained by those skilled in the art based on the embodiments of the present application without creative efforts shall fall within the scope of the present application.

It should be noted that all the directional indications (such as up, down, left, right, front, rear, etc.) in the embodiments of the present application are only used to explain the relative positional relationship, movement, etc. among the components in a certain posture (as shown in the drawings). If the specific posture changes, the directional indication will change accordingly.

In the present application, unless otherwise specifically specified and limited, the terms “connected”, “fixed”, etc. should be understood in a broad sense, for example, “fixed” can be a fixed connection, a detachable connection, or be integrated as a whole; “connected” can be a mechanical connection or an electrical connection, or can be directly connected, or indirectly connected through an intermediate medium, or can be the internal communication between two elements or the interaction relationship between two elements. For those skilled in the art, the specific meaning of the above-mentioned terms in the present application can be understood according to specific situations.

Besides, the descriptions defined with “first”, “second”, etc. in the present application are merely for descriptive purposes, and cannot be understood as indicating or suggesting relative importance or impliedly indicating the number of the indicated technical feature. Therefore, the features defined with “first” or “second” can expressly or impliedly include at least one such feature. The meaning of “and/or” appearing in the disclosure includes three parallel schemes. For example, “A and/or B” includes a scheme A, or a scheme B, or both schemes A and B.

In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

A nasal aspirator 100 is introduced in an embodiment of present application.

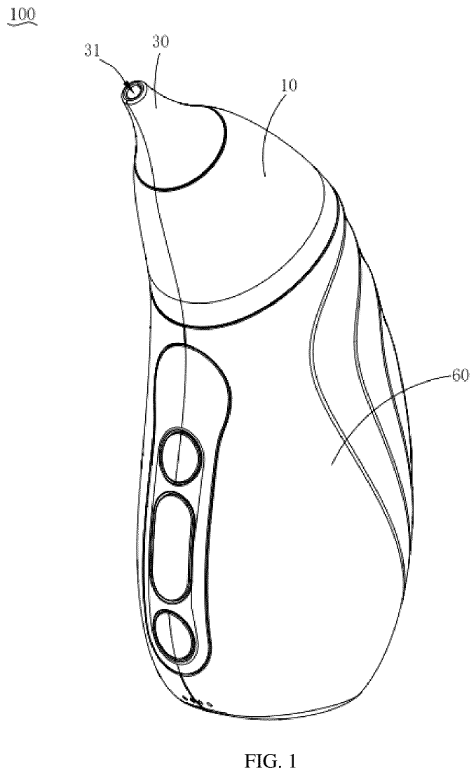

As shown in FIGS. 1-3, the nasal aspirator 100 includes a head portion 10, a reservoir 121, a drainage tube 20, an anti-reflux member 40 and a negative pressure member 50. The head portion 10 is provided with a first cavity 111 inside. The reservoir 121 is connected to the head portion 10 and is disposed in the first cavity 111, the reservoir 121 is provided with a second cavity 1211 inside, and a third cavity 13 is formed between the reservoir 121 and the head portion 10. The drainage tube 20 is connected to the head portion 10 and is extended from a first end of the reservoir 121 into the second cavity 1211, and the drainage tube 20 is provided with a first air channel 21 inside. The anti-reflux member 40 is abutted against a position between the reservoir 121 and the head portion 10 and/or a position between the reservoir 121 and the drainage tube 20. An end of the anti-reflux member 40 close to the drainage tube 20 is extended along an outer wall of the drainage tube 20 and towards a second end of the reservoir 121, and the anti-reflux member 40 is provided with a second air channel 41 which communicates the second cavity 1211 with the third cavity 13. The negative pressure member 50 is communicated with the third cavity 13 and is configured for an air suction to make the third cavity 13 generate negative pressure, and the negative pressure is transmitted from the second air channel 41 and the second cavity 1211 to the first air channel 21.

When the nasal aspirator 100 is used to suck a nasal foreign body such as snot, the head portion 10 is placed into the nasal cavity, the switch of the negative pressure member 50 is turned on, and then the negative pressure member 50 sucks air. Both the first air channel 21 and the second air channel 41 which are communicated with the negative pressure member 50 generate the negative pressure, and the nasal foreign body in the nasal cavity is detached from the nasal cavity and enters the first air channel 21 due to the negative pressure in the first air channel 21. The nasal foreign body entering into the first air channel 21 flows into the reservoir 121 through the first air channel 21 due to the gravity and the negative pressure in the first air channel 21, finally the nasal foreign body is stored in the reservoir 121 due to the gravity, thereby realizing a function that the nasal foreign body is sucked by the nasal aspirator 100.

When the nasal aspirator 100 is accidentally knocked over, it gradually changes from a vertical posture to a horizontal posture, the nasal foreign body in the reservoir 121 will first flow along an inner wall of the reservoir 121. One end of the anti-reflux member 40 abuts against the reservoir 121, so the nasal foreign body cannot flow out of the reservoir 121 from the end of the anti-reflux member 40 that abuts against the reservoir 121, then the nasal foreign body will flow along the anti-reflux member 40 to the other end of the anti-reflux member 40. Since the other end of the anti-reflux member 40 is extended towards a second end of the reservoir 121, so that the nasal foreign body flowing along the anti-reflux member 40 cannot cross the end of the anti-reflux member 40 close to the drainage tube 20, thereby preventing the nasal foreign body from flowing out of the reservoir 121, the reliability of use is high. The nasal foreign body cannot reflux into the first air channel 21 and blockage of the first air channel 21 is prevented, to improve the effect of sucking the nasal foreign body. Meanwhile, neither the nasal foreign body enters the second air channel 41 nor the air suction channel 52, which prevents the nasal foreign body from entering and blocking the second air channel 41 and the air suction channel 52, thereby preventing the negative pressure member 50 from abrasion, to increase the service life of the product. In addition, the second air channel 41 that communicates the second cavity 1211 with the third cavity 13 is opened on the anti-reflux member 40, there is no need to open a groove for passing air on the cavity wall of the first cavity 111, which facilitates the cleaning of the second air channel 41. The anti-reflux member 40 may be a flexible part, specifically a silicone part.

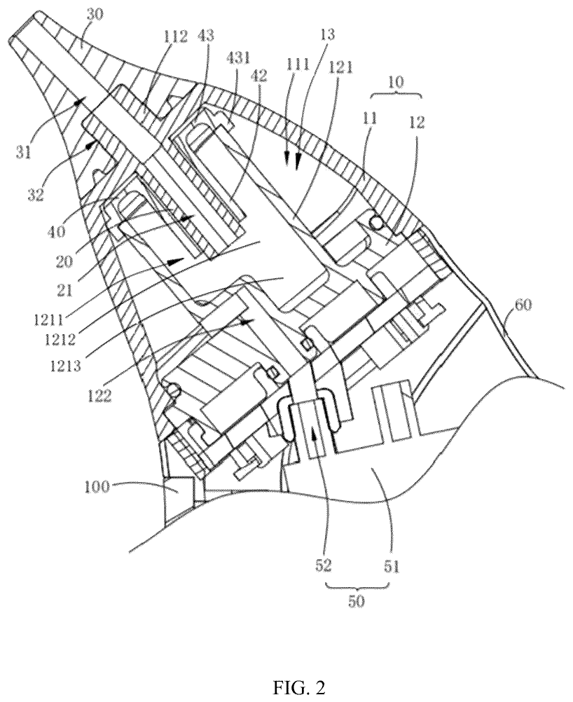

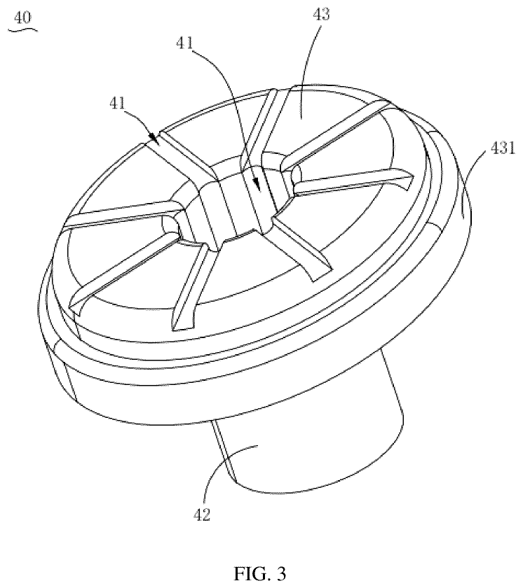

As shown in FIGS. 2 and 3, in an embodiment, the anti-reflux member 40 includes a sleeve 42 and an abutting ring 43. The sleeve 42 is provided around and abutted against a periphery of the reservoir 121. The sleeve 42 is extended towards the second end of the reservoir 121. An inner edge of the abutting ring 43 is connected to the sleeve 42, and an outer edge of the abutting ring 43 is abutted against the reservoir 121. Both the sleeve 42 and the abutting ring 43 are provided with the second air channel 41. The abutting ring 43 realizes an abutment with the reservoir 121, and the nasal foreign body flowing along the abutting ring 43 is blocked by the sleeve 42, the structural design is simple and reasonable.

In an embodiment, a side of the abutting ring 43 away from the base 12 is abutted against the upper housing 11, and the reservoir 121 has more space for storage and more storage capacity, to improve the flexibility of using the nasal aspirator 100.

In an embodiment, a sealing ring 431 is further provided and is connected to the outer edge of the abutting ring 43, the sealing ring 431 is enclosed and abutted to the periphery of the reservoir 121. By providing the sealing ring 431, the nasal foreign body is further prevented from flowing out from the outer edge of the abutting ring 43, to further improve the reliability of using the nasal aspirator 100.

In an embodiment, both the sleeve 42 and the drainage tube 20 are extended towards the second end of the reservoir 121, and an end of the drainage tube 20 close to the second end of the reservoir 121 is extended out of an end of the sleeve 42 close to the second end of the reservoir 121. Therefore, the nasal foreign body can be more reliably retained in the reservoir 121, to further improve the reliability of using the nasal aspirator 100.

In an embodiment, the nasal aspirator 100 includes a plurality of the second air channels 41, and the plurality of the second air channels 41 are provided at intervals along a periphery of the anti-reflux member 40. By opening the plurality of the second air channels 41, a greater negative pressure is generated, which improves the working efficiency of the nasal aspirator 100.

In an embodiment, the reservoir 121 includes a first reserve portion 1212 and a second reserve portion 1213. The second reserve portion 1213 is connected to a side of an end of the first reserve portion 1212 away from the drainage tube 20, and the second reserve portion 1213 is extended away from the drainage tube 20. The nasal foreign body is more likely to stay in the second reserve portion 1213 when the nasal aspirator 100 is tilted, which further prevents the nasal foreign body from flowing out of the reservoir 121 and further improves the reliability of using the nasal aspirator 100.

In an embodiment, the reservoir 121 is provided at a middle of the first cavity 111, and the drainage tube 20 corresponds to a center of the reservoir 121. Compared with the eccentric setting of the drainage tube 20, the setting of the drainage tube 20 in the center of the reservoir 121 ensures that the nasal foreign body will not flow out of the reservoir 121 when the nasal aspirator 100 is tilted at any angle, which improves the reliability of using the nasal aspirator 100.

In an embodiment, the head portion 10 includes an upper housing 11 and a base 12. The first cavity 111 is formed between the upper housing 11 and the base 12. The negative pressure member 50 is connected to the base 12. An air hole 122 is formed at the base 12. The negative pressure member 50 is communicated with the third cavity 13 through the air hole 122. The nasal aspirator 100 further includes a nozzle 30, the nozzle 30 is mounted on a side of the upper housing 11 away from the first cavity 111. The nozzle 30 is provided with an air inflow channel 31 communicated with the first air channel 21. The setting of the nozzle 30 makes it more convenient to reach into the nasal cavity and to suck the nasal foreign body, to improve the convenience of using the nasal aspirator 100.

As shown in FIG. 2, in an embodiment, the nozzle 30 is detachably mounted on the upper housing 11, which facilitates a replacement of the nozzle 30 and improves the convenience of using the nasal aspirator 100. Specifically, the upper housing 11 is provided with a mounting base 112 on a side of the upper housing 11 away from the first cavity 111, and an insertion slot 32 is opened in the nozzle 30 for mating with the mounting base 112, which realizes a detachable installation of the nozzle 30 on the upper housing 11, and the structural design is simple and reasonable.

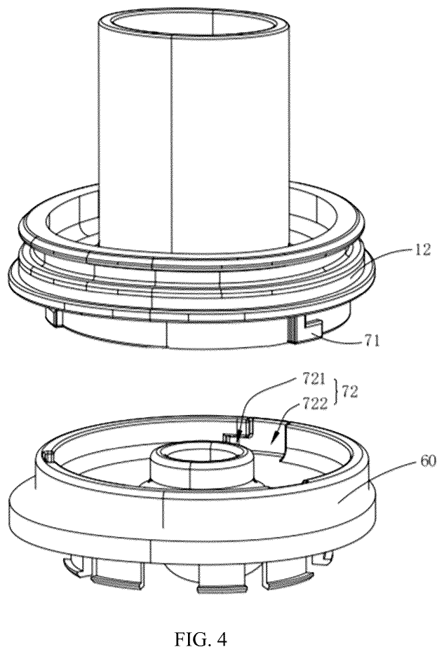

As shown in FIG. 4, in an embodiment, the nasal aspirator 100 further includes a main body 60, the base 12 is detachably mounted on the main body 60 by a connecting structure 70. The negative pressure member 50 is provided inside the main body 60. The negative pressure member 50 includes an air pump 51 and a suction pipe, an air suction channel 52 is formed inside the suction pipe. The air pump 51 is communicated with the air suction channel 52 for the air suction to make the air inflow channel 31 generate the negative pressure. The base 12 mounted detachably on the main body 60 facilitates the maintenance of the internal structure of the main body 60, which improves the convenience of using the nasal aspirator 100.

In an embodiment, the connecting structure 70 includes a clamping block 71 and a clamping groove 72. The clamping block 71 is provided on the base 12, and the clamping groove 72 is provided on the main body 60. The clamping groove 72 includes an insertion portion 721 and a clamping portion 722 communicated with the insertion portion 721. The clamping block 71 is configured to insert from the insertion portion 721 and rotate to clamp with the clamping portion 722. When the base 12 needs to be installed on the main body 60, the clamping block 71 inserts from the insertion portion 721 and rotates to the clamping portion 722, then the clamping block 71 abuts against the groove wall of the clamping portion 722 to realize a clamping of the clamping block 71 with the clamping groove 72, to realize an installation of the base 12. When the base 12 needs to be disassembled, the clamping block 71 rotates in the reverse direction to reach into the insertion portion 721, then the clamping block 71 can be taken out from the insertion portion 721, that is, the base 12 can be disassembled. The structure is simple, the disassembly and installation are easy, to further improve the convenience of using the nasal aspirator 100.

The above-mentioned embodiments are only some embodiments of the present application, and are not intended to limit the scope of the present application. Any equivalent structure conversion made with reference to the description and the accompanying drawings of the present application, direct or indirect application in other related technical fields, should all fall in the scope of the present application.

What is claimed is:

1. A nasal aspirator, comprising:
a head portion provided with a first cavity inside;
a reservoir connected to the head portion and provided in the first cavity, wherein the reservoir is provided with a second cavity inside, and a third cavity is formed between the reservoir and the head portion;
a drainage tube connected to the head portion and extended from a first end of the reservoir into the second cavity, wherein the drainage tube is provided with a first air channel inside;
an anti-reflux member abutted against a position between the reservoir and the head portion and/or a position between the reservoir and the drainage tube, wherein an end of the anti-reflux member close to the drainage tube is extended along an outer wall of the drainage tube and towards a second end of the reservoir, and the anti-reflux member is provided with a second air channel which communicates the second cavity with the third cavity; and
a negative pressure member communicated with the third cavity and configured for air suction, to make the third cavity generate negative pressure, wherein the negative pressure is transmitted from the second air channel and the second cavity to the first air channel.

2. The nasal aspirator according to claim 1, wherein the anti-reflux member comprises a sleeve and an abutting ring;
the sleeve is provided around and abutted against a periphery of the reservoir;
the sleeve is extended towards the second end of the reservoir;
an inner edge of the abutting ring is connected to the sleeve, and an outer edge of the abutting ring is abutted against the reservoir; and
both the sleeve and the abutting ring are provided with the second air channel.

3. The nasal aspirator according to claim 2, wherein both the sleeve and the drainage tube are extended towards the second end of the reservoir, and an end of the drainage tube close to the second end of the reservoir is extended out of an end of the sleeve close to the second end of the reservoir.

4. The nasal aspirator according to claim 1, wherein the nasal aspirator comprises a plurality of the second air channels, and the plurality of the second air channels are provided at intervals along a periphery of the anti-reflux member.

5. The nasal aspirator according to claim 1, wherein the reservoir comprises a first reserve portion and a second reserve portion, the second reserve portion is connected to a side of an end of the first reserve portion away from the drainage tube, and the second reserve portion is extended away from the drainage tube.

6. The nasal aspirator according to claim 1, wherein the reservoir is provided at a middle of the first cavity, and the drainage tube corresponds to a center of the reservoir.

7. The nasal aspirator according to claim 1, wherein the head portion comprises an upper housing and a base, the first cavity is formed between the upper housing and the base;
the negative pressure member is connected to the base;
an air hole is formed at the base;
the negative pressure member is communicated with the third cavity through the air hole;
the nasal aspirator further comprises a nozzle, the nozzle is mounted on a side of the upper housing away from the first cavity; and
the nozzle is provided with an air inflow channel communicated with the first air channel.

8. The nasal aspirator according to claim 7, wherein the nozzle is detachably mounted on the upper housing.

9. The nasal aspirator according to claim 7, wherein the nasal aspirator further comprises a main body, the base is detachably mounted on the main body by a connecting structure;
the negative pressure member is provided inside the main body;
the negative pressure member comprises an air pump and a suction pipe, and an air suction channel is formed inside the suction pipe; and
the air pump is communicated with the air suction channel for the air suction, to make the air inflow channel generate the negative pressure.

10. The nasal aspirator according to claim 9, wherein the connecting structure comprises a clamping block and a clamping groove,
the clamping block is provided on the base, and the clamping groove is provided on the main body;
the clamping groove comprises an insertion portion and a clamping portion communicated with the insertion portion; and
the clamping block is configured to insert from the insertion portion and rotate to clamp with the clamping portion.
